(19) United States
(12) Patent Application Publication
(10) Pub. No.: US 2009/0148512 A1
(43) Pub. Date: Jun. 11, 2009

(54) NOVEL USES OF CHLORAMPHENICOL AND ANALOGOUS THEREOF

(75) Inventor: Arthur P. Bedrosian, Pomona, NY (US)

Correspondence Address: FOX ROTHSCHILD LLP;PRINCETON PIKE CORPORATE CENTER, 2000 Market Street, Tenth Floor, Philadelphia, PA, 19103, US

(73) Assignee: LANNETT CO INC, Philadelphia, PA (US)

(21) Appl. No.: 12/104,766

(22) Filed: Apr. 17, 2008

Related U.S. Application Data
(60) Provisional application No. 61/012,311, filed on Dec. 7, 2007.

(51) Int. Cl. A61K 31/165; A61K 9/20; A61K 9/48; A61P 31/04

(52) U.S. Cl. 424/451; 514/628; 424/464

(57) ABSTRACT

A method for reducing the resistance of an MRSA bacterium to an antibiotic selected from the group consisting of vancomycin and methicillin comprising administering to a patient in need thereof an effective amount of chloramphenicol or analogues thereof. The invention is also directed to chloramphenicol containing pharmaceutical compositions.

### FIELD OF INVENTION

The present invention relates generally to the field of bacteriology, antimicrobial, antibiotics and antibacterial agents. particularly, it provides novel methods of use, kits and combination of antibiotic agents. More particularly, the instant invention is directed to novel methods of using antibiotics against resistant gram positive bacteria.

### BACKGROUND OF THE INVENTION

The present invention was developed in part from a detailed analysis of the scientific literature and an assimilation of known, but previously unconnected, facts. Certain of the publications in this area are described in the following sections and incorporated herein in their entirety.

Antibiotics were introduced into the medical practice in early half of the 1900s. The use of such agents dramatically improved clinical management of infectious conditions. However, irresponsible uses of broad spectrum antibiotics have led to a rapid rise in resistant strains of bacteria and therefore incidences of hard to treat infections. The continuing search for new and effective antibiotics and antibacterial agents motivate the researches to revisit the use of older antibiotics to combat the surge in bacterial infections.

The development of antibiotic resistance is now a reality and an ongoing global treat. Increase incidences of bacterial resistance have serious and life-threatening circumstances. One of ordinary skill in the art can appreciate the social risk associated with the evolution of bacterial resistance across bacterial strains. Such strains as vancomycin-resistant enterococci, vancomycin-resistant Staphylococcus aureus, methicillin-resistant Staphylococcus aureus, penicillin-resistant Staphylococcus pneumoniae and pneumococci have only had a few therapeutic options in recent years.

One such strains of bacteria that has developed immunity against nearly all antibiotics is Staphylococcus aureus. Staphylococcus aureus is a major cause of potentially life-threatening infections acquired in health care and community settings. A dramatic increase in the number of health care-associated infections due to methicillin-resistant Staphylococcus aureus (MRSA) in the 1980s-1990s and the recent emergence of MRSA in community-associated infections highlight the success of this strains of bacteria and its ability to adapt to the unfriendly environments.

MRSA is a strain of Staphylococcus aureus that is resistant to all penicillinase-resistant penicillins and cephalosporins. Such strain is usually resistant to other antibiotics including but not limiting to aminoglycosides, tetracyclines, clindamycin and macrolide antibiotics. One of skill in the art can appreciate the fact that MRSA is a significant problem in almost every major medical center in the U.S.

Without being bound to any theories, mechanism of resistance for MRSA is due to the altered penicillin-binding proteins of MRSA. Beta lactam antibiotics (e.g. penicillins and cephalosporins) damage bacteria by inactivating penicillin binding proteins (PBPs) which are essential in the assembly of their bacterial cell wall. Acquisition of the mecA gene within a plasmid by Staphylococcus aureus codes for the mutated penicillin binding protein termed PBP2a. Such binding protein has a low affinity for beta-lactam antibiotics virtually providing a complete resistance to all penicillin antibiotics.

Research indicates that the mechanism of resistance for MRSA continues to evolve against other antibiotics. Resistance of MRSA to nearly all antibiotic classes has left vancomycin as the only viable option for treatment of serious MRSA-associated infections in the United States.

Glycopeptides such as vancomycin have traditionally provided effective therapy against most multidrug-resistant strains of Staphylococcus aureus. Although vancomycin resistance was first reported for enterococci in mid 80s, the first clinical isolates of high-level vancomycin-resistant Staphylococcus aureus (VRSA) was not isolated until early 2000. Vancomycin is a bactericidal antibiotic that inhibits the synthesis of the cell wall in sensitive bacteria by binding with high affinity to the D-alanyl-D-alanine terminus of cell wall precursor units.

Enterococcal resistance to vancomycin is the result of alteration of D-alanyl-D-alanine target to D-alanyl-D-lactate or D-alanyl-D-serine, both of which dramatically decrease the affinity to vancomycin. It is believed that the mechanism of VRSA resistance is due to the transfer of such mechanism from enterococcal resistant strains to staphylococcal. It has been recognized in the laboratory community that VRSA isolates identified in the U.S. contain mecA and vanA genes mediating oxacillin and vancomycin resistance, respectively.

The genetic exchange of antimicrobial resistance determinants among enterococci and staphylococci is well documented. (see Firth et al, 2000. Genetics: accessory elements and genetic exchange, p. 326-338. Francia et al 2002. Mol. Microbiol. 45:375-395). The resistance genes are typically found on conjugative plasmids or transposons. One requirement for the conjugative transfer of mobile genetic elements is cell-to-cell contact between donor and recipient.

To facilitate this contact, enterococci have highly evolved conjugative systems that are responsible for the dissemination of antimicrobial resistance and virulence factors. These systems include the secretion of bacterial sex pheromones, small peptides that induce a mating response resulting in the aggregation or clumping of the cells. (see Stewart et al, 2001, Lancet 358: 135-138).

One of ordinary skill in the art would know that cell-to-cell contact occurs naturally in microbial biofilms. Microbial cells attached to a surface produce an extracellular polymeric substance that supports a highly structured microbial community. Cells within this matrix have increased tolerance to antimicrobial agents, making it difficult or impossible to eradicate the biofilm once it becomes established. (see Donlan et al, 2002, Clin Microbiol. Rev. 15: 167-193). Many species of microorganisms colonize and form biofilms on a variety of indwelling medical devices such as nephrostomy tube, foley catheter, intravenous (IV) catheters or other types of IV lines, feeding tubes and dialysis access ports.

According to interpretive criteria defined by the National Committee for Clinical Laboratory Standards, the minimal inhibitory concentrations (MICs) of vancomycin for a susceptible bacterial isolates is usually below 8 μg per milliliter. Using the National Committee for Clinical Laboratory Standards broth microdilution reference method, a Staphylococcus aureus isolate with reduced susceptibility to vancomycin is determined VRSA when the MIC is greater than 32 μg/mL or in some cases equal to 64 μg/mL. Comparison of the isolate with MRSA isolated obtained and VRSA has also suggested that the S. aureus with reduced susceptibility to vancomycin emerges from the MRSA strain with which patients are infected.

Despite diagnostic advances, the only way to know if a patient has VRSA is to do a culture sensitivity in a collected patient specimen. Symptoms of chronic VRSA infection include dry, rough, scaly skin around the infected area, fatigue, fever, nausea, and vomiting, pain and redness at the infection site and/or swelling or drainage at the infected sites. Atypical phenotypic characteristics of culture, including weak or negative latex-agglutination test results, weak or negative-slide coagulase test results, heterogeneous morphologic features, slow rate of growth, and vancomycin susceptibility (by disk diffusion test) can usually be observed. (see Rotune et al, Emerg Infec Dis, 1999, January-February 5(1):147-9).

One of ordinary skill in the art can appreciate that essentially everyone is at risk of VRSA. However, patients who have received vancomycin for an infection, or have at some point a colony of MRSA are more likely to develop VRSA type infections. At risk are patients who have had surgery, are in the intensive care unit (ICU) or have been in the ICU, are a dialysis or diabetic patient, have a indwelling medical device, tube or IV lines, have been in close contact with someone who has had VRSA, and have taken broad-spectrum antibiotics for conditions that are viral.

Even though the public health response to identification of the VRSA infection is ongoing, the use of proper infection-control practices and appropriate antimicrobial agent management can help limit the emergence and spread of antimicrobial-resistant microorganisms MRSA and VRSA. One of ordinary skill in the art can appreciate that there is a grave need in the art to develop effective antibiotic regimens to ward off the emergence of MRSA and VRSA. At least one aspect of this invention is to provide alternative antibiotic regimens against MRSA and VRSA infections. The envisioned regimen employs older generation antibiotics, newer analogues thereof in single therapeutic regimen as well as their combination with other suitable antibiotics.

### SUMMARY OF THE INVENTION

The present invention seeks to overcome the drawbacks inherent in the prior art by providing new methods, compositions, regimens and kits for treating and/or reducing bacterial resistance to antimicrobials and antibiotics. More specifically, the present invention is directed to effective management of VRSA or MRSA infections in patient susceptible or at risk of developing such infections. The invention rests in the surprising use of a new chloramphenicol oral formulation by itself or in conjunction with a second suitable agent.

At least one aspect of this invention embraces the use of antibiotics such as chloramphenicol alone or in combination with suitable antibiotics preferably linezolid, minocycline, quinupristin-dalfopristin, rifampin, and trimethoprim-sulfamethoxazole. In this aspect of the invention, the formulated composition can be in an immediate release, sustain release or delayed release dosage form.

In another embodiment of the invention, the inventors embrace an individualized therapeutic regimen for VRSA isolates with MICs ranged from 32 to >128 μg/ml. In a more preferred embodiment, the inventors envision a use of aggressive therapeutic regimen including a combination of targeted chloramphenicol treatment in conjunction with monitoring parameters necessary to optimize individualized care. The inventors believe that VRSA isolates are generally resistant to aminoglycosides, fluoroquinolones, macrolides, penicillin, and tetracycline but remained susceptible to chloramphenicol, linezolid, rifampin, and trimethoprim-sulfamethoxazole. Accordingly, in the most preferred embodiment, a combination of suitable antibiotics are employed to effectively reduce the risk of developing or treat MRSA and/or VRSA infections.

In another aspect of the invention, inventors teach new oral formulations containing at least up to 75% (w/w) of an antibiotic or antibacterial compound, 10-50% (w/w) of a diluent, 1-30% (w/w) of a binder, 0-20% (w/w) of a superdisintegrant, 0.5-20% (w/w) of a lubricant and other suitable pharmaceutically acceptable ingredients. In a more preferred embodiment, inventors envision tablets or capsules containing chloramphenicol or an analogue thereof alone or in combination with a second agent that would improve the final clinical outcome.

In at least one embodiment of the instant invention, the inventor has prepared a capsule comprising chloramphenicol in amounts of about 50-75% w/w, a binder such as lactose in amounts of about 15-30% w/w, and a lubricant such as a vegetable oil in amounts of about 2.5-5% w/w.

In a more preferred embodiment, the capsule comprises levochloramphenicol in amounts of about 70% w/w, lactose in amounts of about 27% w/w and a vegetable oil in amounts of about 3%.

### BRIEF DESCRIPTION OF THE DRAWINGS

FIG. 1. illustrates the activity of chloramphenicol and other antibiotics against Staphylococcus aureus collected in North America and Europe in 2005.

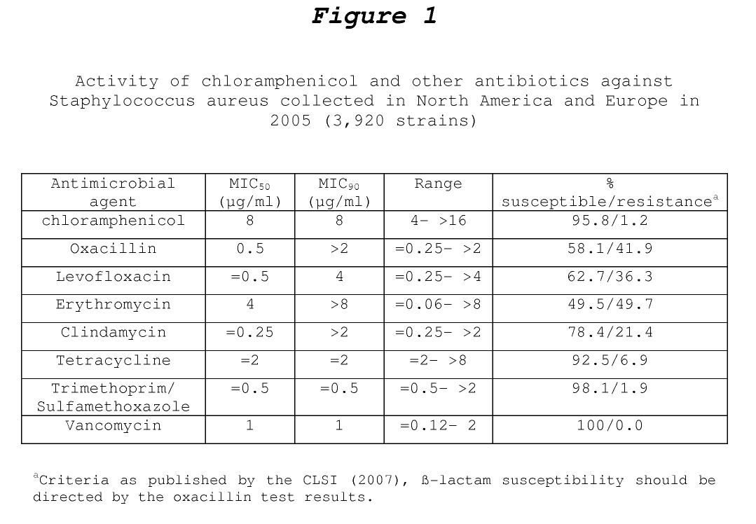

### DETAILED DESCRIPTION OF THE INVENTION

The terms “microorganism,” “infectious pathogen,” “bacteria” and “bacterium” are used for simplicity and it will be understood that the invention is suitable for use against a population of microorganisms, i.e., “bacteria”.

The microorganism, e.g., bacterium, or population thereof, may be contacted either in vitro or in vivo. Contacting in vivo may be achieved by administering to an animal (including a human patient) that has, or is suspected to have a microbial or bacterial infection, a therapeutically effective amount of pharmacologically acceptable antibiotic agent formulation alone or in combination with a therapeutic amount of a pharmacologically acceptable formulation of a second agent effective to inhibit the growth of the pathogen, e.g., another antibiotic or an agent that improves efficacy of chloramphenicol. The invention may thus be employed to treat both systemic and localized microbial and bacterial infections by introducing the combination of agents into the general circulation orally or parentally or by applying the combination, topically to a specific site, such as a wound or burn, or to the eye, ear or other site of infection.

By the term “antibiotic,” “antibiotic containing drug,” “antibiotic or antimicrobial compositions,” it is meant formulations that contain at least one agent that has bactericidal or bacteriostatic activity against MRSA or VRSA. Further, by the term the “active drug” it is meant all form of such drugs that can yield therapeutic results including but not limiting to enantiomers, stereochemical isomers, levo or dextro form of such compounds, hydrates, solvates, tautomers and pharmaceutically acceptable salts thereof.

The term “oral formulation” refers to medicinal dosages in the form of tablet, capsule, lozenges, trochees, powders, syrups, elixirs, aqueous suspension or solutions that contain up to 75% of an active ingredient and can be either in the form of sustained release, delayed release or non-sustained release such as immediate release formulations, or chewable tablets. The most preferred of such formulations are in the form of a tablet or a capsule.

The term “binder” or “binding agent” refer to conventional pharmaceutically acceptable binding agents such as cellulose derivatives, e.g. ethyl cellulose, hydroxyethyl cellulose, carboxymethyl cellulose, methyl cellulose, hydroxypropylmethyl cellulose, or gelatin, starch, Polyvinyl alcohols, gum Arabic, glucose, alginates, polyacrylic acids.

The term “flow promoting agents” are directed to agents that improve the flow of the tablet ingredients such as colloidal silicon dioxide, talcum.

The term “superdisintegrants” refer to croscarmellose sodium, sodium starch glycolate, and L-hydroxypropyl cellulose.

The term “lubricant” refers to such compounds as magnesium stearate, calcium stearate, steric acid, suitable oils or agents that are conventionally used to provide such function during the process of preparing a tablet, capsule or a sustain released matrix.

An “effective amount of an antimicrobial agent or antibiotic” means an amount, or dose, given or prescribed to achieve therapeutic MIC concentration. Such ranges are well established in routine clinical practice, or yet can be determined by those of skill in the art for bolus, baseline and maintenance doses. Appropriate oral and parenteral doses and treatment regimens are further detailed herein.

As this invention provides for enhanced microbial and/or bacterial killing, it will be appreciated that effective amounts of an antimicrobial agent or antibiotic may be used that are lower than the standard doses previously recommended when the antimicrobial or antibiotic is administered alone.

The “second agents” for use in the invention are generally a drug that enhances or synergizes the activity of chloramphenicol or is able to treat infections directly. The second agent inhibitors should be used in amounts effective to inhibit the growth of a microorganism or bacteria, as exemplified by an amount effective to reach suitable steady state serum or other tissue concentrations.

In addition to the present disclosure and the references specifically incorporated herein, there is considerable scientific literature concerning treating MRSA or VRSA that may be utilized in light of the inventors' discovery. Accordingly, the compositions of the instant invention may be effectively combined with other antibiotics and other antimicrobial agents to achieve a bacteriocidal or bacteriostatic activity at a site of interest.

Naturally, in confirming the optimal therapeutic dose, first animal studies and then clinical trials would be conducted, as is routinely practiced in the art. Animal studies are common in the art and are further described herein and in publications such as Lorian (1991, pp. 746-786, incorporated herein by reference) and Cleeland & Squires (incorporated herein by reference, from within the Lorian text).

In a clinical trial, the therapeutic dose would be determined by maximizing the benefit to the patient, whilst minimizing any side-effects or associated toxicities. Throughout the detailed examples, various therapeutic ranges are listed. Unless otherwise stated, these ranges refer to the amount of an agent to be administered orally.

In optimizing a therapeutic dose within the ranges disclosed herein, one would not use the upper limit of the range as the starting point in a clinical trial due to patient heterogeneity and drug toxicity, i.e. aplastic anemia. Starting with a lower or mid-range dose level, and then increasing the dose will limit the possibility of eliciting a toxic or untoward reaction in any given patient or subset of patients. The presence of some side-effects or certain toxic reactions per se would not, of course, limit the utility of the invention, as it is well known that most beneficial drugs also produce a limited amount of undesirable effects in certain patients. Also, a variety of means are available to the skilled practitioner to counteract certain side-effects, such as using vitamin supplementations, hydration modifying antibiotic regimens, e.g. frequency, intervals, or reducing or discontinuing the offending agent.

It is important to note that at least one aspect of the instant invention concerns the new and surprisingly effective use of compounds, already known to have certain functional properties, alone or in combination with second or third antimicrobial agents and/or antibiotics. Such compounds can be used in their racemate, pure, isolated stereochemical, enantiomeric or diastereomeric forms.

Zak & Sande (1981) reported on the correlation between the in vitro and in vivo activity of a 1000 compounds that were randomly screened for antimicrobial activity. The important finding in this study is that negative in vitro data is particularly accurate, with the negative in vitro results showing more than a 99% correlation with negative in vivo activity.

This is meaningful in the context of the present invention as one or more in vitro assays will be conducted prior to using any given combination in a clinical setting. Any negative result obtained in such an assay will thus be of value, allowing efforts to be more usefully directed.

Chloramphenicol is an antibiotic produced by Streptomyces venezuelae, an organism first isolated in 1947 from soil samples collected in Venezuela having the following structure:

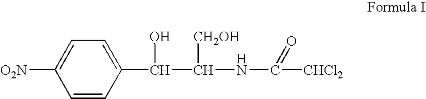

Chloramphenicol is primarily a bacteriostatic antibiotic which exerts it action by inhibiting protein synthesis in bacteria. Chloramphenicol readily penetrates bacterial cells and acts primarily by binding reversibly to the 50S ribosomal subunits near the site of action of macrolide antibiotics and clindamycin which it inhibits competitively. Although binding of tRNA at the codon recognition site on the 30S ribosomal unit is undisturbed, chloramphenicol appears to prevent the binding of the amino acid containing end of the aminoacyl tRNA to the acceptor site on the 50S ribosomal unit. Accordingly, the interaction between peptidyltransferase and its amino acid substrate can not occur and peptide bond formation is inhibited.

Chloramphenicol possess a wide spectrum of antimicrobial activity. Strains are considered sensitive if they are inhibited by concentration of 8 μg/ml or less, except N. gonorrhea, S. pneumoniae and H. Influenza which have lower MIC breakpoint. Even though, the prevalence of chloramphenicol resistance of staphylococci has increased, Chloramphenicol and its analogues remain the most promising alternative to MRSA and VRSA infections.

To reduce the resistance of a microorganism to an antimicrobial agent, as exemplified by reducing the resistance of a bacterium to an antibiotic, or to kill a microorganism or bacterium, one would generally contact the microorganism or bacterium with an effective amount of the antibiotic or antimicrobial agent alone or in combination with an amount of a second agent effective to inhibit the growth of the microorganism or the bacterium. In terms of killing or reducing the resistance of a susceptible bacterium, one of ordinary skill in the art would contact the bacterium with an effective amount of an Chloramphenicol alone or in combination with an amount of a second agent effective that can inhibit the bacterial multiplication, synthesis and/or maturation at the site of interest.

The inventors contemplate that effective use of chloramphenicol therapy alone or in combination with other suitable antibiotic regimes can play an important role in effective management of MRSA and VRSA infections. For example, the data listed in Table I-VI elaborate on high degree of sensitivity of various common bacteria to chloramphenicol. Using the antibiotics listed herein, amongst others, in combination with chloramphenicol would improve the clinical outcome of patient suffering from a MRSA or VRSA infections.

TABLE I
In vitro activity of chloramphenicol and metronidazole tested
against 25 clinical strain of C. difficle.

| Antimicrobial | MIC₅₀ | | | % susceptible/ |
|---|---|---|---|---|
| agent | (μM) | MIC₉₀ (μM) | Range | % resistant |
| Chloramphenicol | 4 | 16 | 2-32 | 88.0/8.0 |
| Metronidazole | 0.25 | 0.5 | 0.12-0.5 | 100/00 |

TABLE II
In vitro activity of chloramphenicol and other antibiotics against
tested MRSA collected in North America and Europe in 2005
(1,644 strains)

| Antimicrobial | MIC₅₀ | MIC₉₀ | | % susceptible/ |
|---|---|---|---|---|
| agent | (μg/ml) | (μg/ml) | Range | resistance^{a} |
| chloramphenicol | 8 | 8 | ≦2->16 | 91.5/1.9 |
| Levofloxacin | >4 | >4 | ≦0.5->4 | 20.6/77.4 |
| Erythromycin | >8 | >8 | 0.12->8 | 10.2/89.2 |
| Clindamycin | ≦0.25 | >2 | ≦0.25->2 | 54.6/45.3 |
| Tetracycline | ≦2 | >8 | ≦2->8 | 88.8/10.3 |
| Trimethoprim/ | ≦0.5 | ≦0.5 | ≦0.5->2 | 96.6/3.4 |
| Sulfamethoxazole | | | | |
| Vancomycin | 1 | 1 | 0.25-2 | 100/0.0 |

TABLE III
In vitro activity of chloramphenicol and other antibiotics
against tested MRSA collected in North America in 2005 (1,158 strains)

| Antimicrobial | MIC₅₀ | MIC₉₀ | | % susceptible/ |
|---|---|---|---|---|
| agent | (μg/ml) | (μg/ml) | Range | resistance^{a} |
| chloramphenicol | 8 | 8 | ≦2->16 | 92.3/0.4 |
| Levofloxacin | >4 | >4 | ≦0.5->4 | 26.4/71.7 |
| Erythromycin | >8 | >8 | 0.12->8 | 4.8/94.8 |
| Clindamycin | ≦0.25 | >2 | ≦0.25->2 | 55.5/44.3 |
| Tetracycline | ≦2 | ≦2 | ≦2->8 | 92.5/7.0 |
| Trimethoprim/ | ≦0.5 | ≦0.5 | ≦0.5->2 | 97.8/2.2 |
| Sulfamethoxazole | | | | |
| Vancomycin | 1 | 1 | 0.25-2 | 100/0.0 |

TABLE IV
In vitro activity of chloramphenicol and other antibiotics against
tested susceptible Staphylococcus aureus (MSSA) collected
in North America and Europe in 2005 (2,276 strains)

| Antimicrobial | MIC₅₀ | MIC₉₀ | | % susceptible/ |
|---|---|---|---|---|
| agent | (μg/ml) | (μg/ml) | Range | resistance^{a} |
| chloramphenicol | 8 | 8 | ≦2->16 | 98.9/0.7 |
| Levofloxacin | ≦0.5 | ≦0.5 | ≦0.5->4 | 93.0/6.7 |
| Erythromycin | o.25 | >8 | ≦0.06->8 | 77.9/21.3 |
| Clindamycin | ≦0.25 | ≦0.25 | ≦0.25->2 | 95.7/4.0 |
| Tetracycline | ≦2 | ≦2 | ≦2->8 | 95.2/4.4 |
| Trimethoprim/ | ≦0.5 | ≦0.5 | ≦0.5->2 | 99.3/0.7 |
| Sulfamethoxazole | | | | |
| Vancomycin | 1 | 1 | ≦0.12-2 | 100/0.0 |

TABLE V
In vitro activity of chloramphenicol and other antibiotics against
tested susceptible Staphylococcus aureus (MSSA) collected
in North America in 2005 (1,232 strains)

| Antimicrobial | MIC₅₀ | MIC₉₀ | | % susceptible/ |
|---|---|---|---|---|
| agent | (μg/ml) | (μg/ml) | Range | resistance^{a} |
| chloramphenicol | 8 | 8 | ≦2->16 | 99.4/0.0 |
| Levofloxacin | ≦0.5 | ≦0.5 | ≦0.5->4 | 93.0/6.6 |
| Erythromycin | o.25 | >8 | ≦0.06->8 | 70.8/28.0 |
| Clindamycin | ≦0.25 | ≦0.25 | ≦0.25->2 | 95.0/4.8 |
| Tetracycline | ≦2 | ≦2 | ≦2->8 | 96.8/2.7 |
| Trimethoprim/ | ≦0.5 | ≦0.5 | ≦0.5->2 | 98.8/1.2 |
| Sulfamethoxazole | | | | |
| Vancomycin | 1 | 1 | ≦0.25-2 | 100/0.0 |

TABLE VI
In vitro activity of chloramphenicol and other antibiotics against
tested susceptible Staphylococcus aureus (MSSA) collected
in Europe in 2005 (1,044 strains)

| Antimicrobial | MIC₅₀ | MIC₉₀ | | % susceptible/ |
|---|---|---|---|---|
| agent | (μg/ml) | (μg/ml) | Range | resistance^{a} |
| chloramphenicol | 8 | 8 | 4->16 | 98.4/1.4 |
| Levofloxacin | ≦0.5 | ≦0.5 | ≦0.5->4 | 93.0/6.8 |
| Erythromycin | o.25 | >8 | ≦0.06->8 | 86.2/13.3 |
| Clindamycin | ≦0.25 | ≦0.25 | ≦0.25->2 | 96.6/3.2 |
| Tetracycline | ≦2 | ≦2 | ≦2->8 | 93.3/6.5 |
| Trimethoprim/ | ≦0.5 | ≦0.5 | ≦0.5->2 | 99.8/0.2 |
| Sulfamethoxazole | | | | |
| Vancomycin | 1 | 1 | ≦0.12-2 | 100/0.0 |

^{a}Criteria as published by the CLSI (2007), β-lactam susceptibility should be directed by the oxacillin test results.

Pharmacokinetic studies have shown that other pharmaceutically effective derivatives of chloramphenicol such as ester derivatives or succinate derivatives are also effective chloramphenicol forms for providing the desired clinical outcome.

The pharmaceutically effective analogous of chloramphenicol of the present invention have the following generic structure:

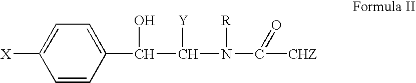

wherein: X is selected from a group consisting of —NO₂, —SO₂, —CN, —SO₂R, —COOR wherein R is a lower alkyl chain having 1-5 carbons atoms, Y is selected from a group consisting of a hydrogen, a lower alkyl or a lower alcohol, R is a hydrogen, a lower alkyl or a lower alcohol, Z is a hydrogen, an alkyl, a halogen, or a halogenated lower alkyl. In the most preferred embodiment, X is a NO₂, Y is a CH₂OH, R is a hydrogen atom, and Z is a Cl₂. the term “lower alkyl” is referred to alkyl chains with one to five carbons atoms.

One of ordinary skill in the art can appreciate that a suitable chloramphenicol analogue formulation would be absorbed rapidly from the GI track and preferably achieve a peak concentrations of 10 to 13 μg/ml within 2 to 3 hours after the administration of a 1 g dose. In at least one embodiment of the instant invention, the chloramphenicol analogue is prepared in oral, topical and injectable forms.

The oral formulation envisioned by the inventors can be prepared both in the form of the active drug itself and the inactive prodrug such as chloramphenicol palmitate. Methods of making such forms of chloramphenicol are described in U.S. Pat. Nos. 2,662,906, 3,652,607 and 3,803,321, the teachings of which are enclosed in their entirety herein.

In another aspect of the instant invention, patient's antibiotic treatment is successfully individualized to reduce the risk of developing MRSA or VRSA infections. In this aspect of the invention, pharmacokinetic and pharmacodynamic concepts are employed to individualize patients antibiotic regimens. In this aspect of the invention, measurements of patients serum or plasma, or other tissue samples for the bacterial sensitivity is correlated with the serum, plasma or other tissue concentrations of antibiotics. Accordingly, one of ordinary skill in the art can combine with the general knowledge known about the infectious condition to influences the disposition of a particular antibiotic regimen by employing kinetic concepts.

In a more preferred embodiment, an antibiotic regimens comprise the steps of establishing compartmental model for distribution of the suitable chloramphenicol analogue to individualize the doses in patients in need of such treatment to establish baseline effects of the chosen antibiotic, establishing duration for development of a resistant strain and employing specific antibiotic holiday periods to reduce risk of developing a MRSA or VRSA infection.

To treat a mammalian subject, such as a human patient, an effective amount of one or more compounds of the present invention, or a pharmaceutically-acceptable salt thereof, is administered to the mammalian subject so as to promote exposure to or contact of infected areas. Effective dosage forms, modes of administration and dosage amounts may be determined empirically, and making such determinations is within the skill of the art. It is understood by the physician, veterinarian or clinician of ordinary skill in the art that the dosage amount will vary with the activity of the particular compound employed, course and/or progression of the disease state, the route of administration, the rate of excretion of the compound, renal and hepatic function of the patient, the duration of the treatment, the identity of any other drugs being administered to the subject, age, size and like factors well known in the medical arts.

The pharmaceutical compositions may also be formulated to suit a selected route of administration, and may contain ingredients specific to the route of administration. Routes of administration of such pharmaceutical compositions are usually split into five general groups: inhaled, oral, transdermal, parenteral and suppository.

As discussed herein, the compounds of the present invention can be administered in such oral dosage forms as tablets, capsules, each of which can be prepared in a sustained release or timed release formulation. The present dosage forms can also be prepared in other forms such as micronized powder, granules, elixirs, tinctures, suspensions, solutions, syrups and emulsions. The compositions of present invention may also be administered in intravenous (bolus or infusion), intraperitoneal, topical (e.g., ocular eye drop), subcutaneous, intramuscular or transdermal (e.g., patch) form. All such dosage forms are well known to those of ordinary skill in the pharmaceutical arts. Again, the ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 200 mg/kg/day, preferably 4 to 150 mg/kg/day, and most preferably 50 to 100 mg/kg/day. For oral administration, the compositions are preferably provided in the form of tablets or capsules containing 5.0, 10.0, 25.0, 50.0, 100, 150, 200, 250 and 500 milligrams of the active ingredient for the symptomatic adjustment of the dosage to the patient to be treated. In a most preferred embodiment, chloramphenicol is in its pure isomeric form creating a more potent formulation than a racemic mixture.

Formulations of the present invention may be administered in a single daily dose, or the total daily dosage may be administered in multiple doses, preferably in 4 divided doses depending on the severity of the infection. At least one aspect of this invention is directed to new oral formulations of chloramphenicol that presents an improved side effect profile, bioavailability and taste. In such embodiment of the instant invention, oral formulations contain from about 0.01 mg to about 500 mg of the active ingredients. In the case of chloramphenicol or its analogues, such amount is preferably from about 25 mg to about 250 mg.

In another embodiment of the instant invention, the therapeutic antibiotic treatment employs the use of a combination of antibiotics. Combinations are generally chosen because an identified pathogen is resistant to inhibition and/or killing by conventional doses of a single antibiotic, but in contrast is susceptible to the combination (Eliopoulos & Moellering, 1991). One particular example of the applicability of the invention is in providing methods and combinations for use in reducing the resistance of MRSA bacteria to vancomycin, ticoplanin, macrolide, aminoglycosides, and penicillin antibiotics, or in enhancing the sensitivity of susceptible strains to such antibiotics. In this case, an chloramphenicol analogue will primarily inhibit MRSA and VRSA growth, while the secondary agent will target the less sensitive bacteria.

In general there are seven basic biochemical mechanisms for naturally-occurring antibiotic resistance have been described (see Davies, 1986), namely alteration of the antibiotic; alteration of the target site; block in the transport of the antibiotic; by-pass of the antibiotic sensitive-step; increasing the level of the inhibited enzyme; the cell is spared the antibiotic-sensitive step by endogenous or exogenous product; and the production of a metabolite that antagonizes action of inhibitor. The same general concepts also apply to microorganisms other than bacteria. (see Lorian, 1991).

This invention therefore encompasses methods to reduce antimicrobial resistance, caused by any of the seven mechanisms described above, using a combination chloramphenicol and a second drug or antibiotic agent that can influence bactericidal activity of chloramphenicol on MRSA or VRSA.

One of ordinary skill in the art can employ accepted mechanisms of antibacterial synergism to reduce the risk of resistance. Such mechanisms include namely, (1) serial or sequential inhibition of a common biochemical pathway (e.g. trimethoprim-sulfamethoxazole); (2) inhibition of protective bacterial enzyme (clavulanic acid plus a β-lactamase-susceptible penicillin); (3) combination of cell wall-active agents (e.g. ampicillin); and (4) use of cell wall-active agents to enhance the uptake of other antimicrobials (e.g. penicillin and streptomycin).

By way of example only, certain infections that may be treated using the invention are systemic and localized infections caused by MRSA and VRSA, such as skin ulcers, nosocomial infections secondary to an implantable device, and UTIs.

In another aspect of this invention, the inventor provides a novel synergistic option for antimicrobial treatment. In such methodologies, chloramphenicol formulation is used in combination with any other antibiotic that can provide at least one of the synergistic mechanism articulated above. Accordingly, a second antibiotic can be chosen to provide at least one such mechanisms of antibacterial synergism. These include an antibiotic compound selected from the group penicillins; first-generation cephalosporin, vancomycin, imipenem, clindamycin, a fluoroquinolone, penicillinase-resistant derivatives thereof, amoxicillin-clavulanic acid, ticarcillin-clavulanic acid, ampicillin-sulbactam; trimethoprim/sulfamethaxazole (TMP-SMX), minocycline gentamicin and/or rifampin, erythromycin, clarithromycin, and azithromycin. Antimicrobial combinations are well known and are most frequently used to provide broad-spectrum empirical coverage in the treatment of patients who are seriously ill.

The inventors envision that MRSA and VRSA infections can be effectively managed by the use of suitable oral chloramphenicol alone or in combination with a secondary agent that can increase susceptibility of MRSA or VRSA to chloramphenicol therapy. More particularly, the inventors envision that the instant methods of using antibiotic drugs will reduce or eliminate resistance to vancomycin and/or methicillin.

Further embodiments of the invention include therapeutic kits that comprise, in suitable container means, a pharmaceutical formulation of at least chloramphenicol, analogous thereof with or without another antimicrobial agent and a pharmaceutical formulation. The antibiotics and inhibitory second agents may be contained within a single container means, or a plurality of distinct containers may be employed.

Although the invention was developed, in part, from a consideration of various biochemical interactions and pathways, an understanding of the precise mechanism by which any given compound functions to reduce resistance in a microorganism, as measured by enhanced killing, is not relevant to practicing the invention. Therefore, at least one aspect of the instant invention is directed to effective management of infections, by using the compounds that inhibit or delay the development of MRSA or VRSA in a given patient, both directly and/or indirectly.

For example, at least one aspect of the invention is directed to methods of treating multipathogenic infections, sepsis, acute respiratory distress syndrome, and even shock comprising administering to the patient in need an effective anti-bacterial amount of a chloramphenicol or analogue thereof, alone or in combination with a secondary agent. In the preferred embodiment of this aspect of the invention, chloramphenicol analogue, or pharmaceutical salt thereof, or composition is administered to a patient, the patient is monitored every 3-5 half-lives for suitable serum concentration and also monitoring of related hemodynamic indices.

For such aspect of the invention, the antibiotic regimen of the present invention in each effective dose is selected with regard to consideration of the resistant strain causing the infection, the severity of infection, the patient's age, weight, sex, general physical condition and the like. The amount of active component required to induce an effective anti-bacterial effect without significant adverse side effects varies depending upon the pharmaceutical composition employed and the optional presence of other components, e.g., antibiotics and the like.

For systemic administration, a therapeutically effective dose can be estimated initially from in vitro assays. For example, a dose can be formulated in animal models to achieve a circulating concentration range in view of the IC₅₀ as determined in cell culture (i.e., the concentration of compounds that is lethal to 50% of a cell culture), the MIC, as determined in cell culture (i.e., the minimal inhibitory concentration for growth) or the IC₁₀₀ as determined in cell culture (i.e., the concentration of chloramphenicol that is lethal to 100% of a cell culture). Such information can be used to more accurately determine optimal doses in animal subjects.

Initial dosages can also be estimated from in vivo data, e.g., animal models, using techniques that are well known in the art. One having ordinary skill in the art could readily optimize administration to humans based on animal data. Based on this information, one may administer the chloramphenicol, or compositions thereof, in single or multiple doses each day. The antibiotic therapy may be repeated intermittently while infections are detectable or even when they are not detectable. Additionally, as provided above, the therapy may be provided alone or in combination with other drugs.

In cases of local administration or selective uptake, the effective concentrations of chloramphenicol may not be related to plasma concentration. One having skill in the art will be able to optimize therapeutically effective local dosages without undue experimentation. For optimal results, the plasma concentrations of chloramphenicol needs to be monitored every 3-5 half lives.

The data presented in the tables of the present specification is another tool to enable the straightforward comparison of raw data with accepted clinical practice and to allow the determination of appropriate doses of combined agents for clinical use.

In another embodiment of the instant invention, the inventors embrace new oral formulations of chloramphenicol wherein tablet compositions of such products comprise a binder, a superdisintegrant, a lubricant and a flow agent. Other oral formulations envisioned by the inventors include oral dry powder formulations consist essentially of chloramphenicol, its analogous or salts thereof. In another aspect of the invention, a new oral formulation containing chloramphenicol can be prepared in capsules, pellet or granulates with high active substance content to achieve higher serum concentration of selected antibiotics.

Accordingly the present invention provides for solid drug formulation comprising finely refined levochloramphenicol, a disintegrant, lubricant and pharmaceutically acceptable diluent. Chloramphenicol generally has an intense bitter taste. In at least one aspect of the instant invention, a suitable formulation is prepared to improve the taste, absorption and delivery of chloramphenicol using diluent, lubricant, disintegrant and flavoring agent.

A suitable disintegrant may be selected from any of the compounds including but not limited to microcrystalline cellulose, starches and starch derivatives alone or in combination with other type of disintegrants generally known as a superdisintegrant, such as croscarmellose, crospovidone and sodium starch glycollate. In some instances it is advantages to use a combination of disintegrants. The amount of disintegrant, or mixture thereof, is from 0 to 25%, preferably from 2.5 to 15%.

The formulations of the invention may contain at least one diluent in order to give sufficient material to tablet and facilitate the compression process used to make tablets. Suitable diluents include microcrystalline cellulose, calcium hydrogen phosphate, and lactose and alike. The amount of diluent is easily ascertainable to those of ordinary skill in the art and can range from 10-50% by weight of the formulation, preferably 20-40% and most preferably about 30%.

The formulations of the invention may contain wetting agents to improve the disintegration and/or dispersion. Suitable wetting agents include dioctyl sodiumsulphosuccinate, polysorbates or sodium lauryl sulphate. The amount of wetting agent is easily known to those of ordinary skill in the art and is usually not more than 0.1% by weight of the formulation.

The formulations of the invention may include lubricants. Suitable compounds include fatty acids such as stearic acid, metal stearates such as magnesium stearate, hydrogenated oils. Examples of such compounds include hydrogenated vegetable or castor oil, talc, and colloidal silicon dioxide. The amounts of lubricants used in the formulation is also easily ascertained by those of ordinary skill in the art and is generally in amounts of up to 5% by weight of the formulation.

In a preferred embodiment the lubricant is a liquid film that can be applied as an auxiliary binder, wherein it can melt and re-solidify during the compaction process, enhancing the bonding capacity of the final oral formulation resulting in a more robust solid dosage form. In a more preferred embodiment, the lubricant is added in the dry state during the last blending operation before compression. In one aspect of this invention, the lubricant can be in combination with talc or an anti-adherent agent. However, the lubricant is preferably substantially free of carbohydrates, proteins and amino acids, starch and starch derivatives and/or any preservatives and has a melting range of between 50-70° C. In the most preferred embodiment, the lubricant is a hydrogenated cotton-seed oil used at a concentration of about 0.5-4% w/w.

Colors, flavors and aromatizing agents may also be included in the formulations. The solid drug formulations may be in the form of a simple mixture of the ingredients which can be filled into sachets that can be emptied into water. Preferably the solid drug formulations are in the form of tablets.

Tablets can be manufactured in several known different ways. In direct compression process a suitable diluent, such as microcrystalline cellulose, selected grades of calcium hydrogen phosphate, or lactose, is chosen to allow the components to be mixed and tabletted.

Capsule formulations may contain the active ingredient and powdered carriers such as lactose, starch, cellulose derivatives, magnesium stearate, stearic acid, vegetable oil and the like. Similar carriers can be also used to make compressed tablets. Both capsules and tablets can also be manufactured as sustained release products to provide for continuous release of medication over a period of hours.

In at least one preferred aspect of the instant invention, chloramphenicol particles are in the form of finely divided powder, particles, granules or pellets having a particle size diameter of 500 nm-2.5 mm, preferably in the ranges of 2000 nm-1.0 mm, and more preferably in the ranges of 100 μm-0.5 mm.

In another aspect of this invention, the chloramphenicol powder, particles, granules or pellets may be coated or combined with pharmaceutically suitable polymer or additive to provide sustained release properties. Such coated forms of chloramphenicol may then be incorporated into a capsule shelling or compressed into a tablet for oral administration.

Many sustained-release formulations are already known, but there is no generally applicable method by which such formulations can be designed. Each formulation is dependent on the particular active substance incorporated therein. The sustained/prolonged release formulations of the instant invention takes into account many factors such as rates of absorption, clearance of the active substance, the activity of excipients and the bioavailability of chloramphenicol derivative. In at least one embodiment of the invention, the powdered particles, granules or pellets are coated with swellable acrylic polymers and/or hydroxylated cellulose derivatives covering substantially the whole surface of said particles, granules or pellets. The methods for preparing a coated particles, granules or pellets are known to those of ordinary skill in the art.

Suitable polymers employed for this aspect of the invention include carboxypolymethylenes (e.g. carbomers), hydrophobic or hydrophilic polymers. Suitable hydrophobic polymers include for example polyvinyl chloride, ethyl cellulose, polyvinyl acetate and acrylic acid copolymers, such as Eudragiths. Suitable hydrophilic polymer include hydroxypropyl methylcellulose, hydroxypropyl cellulose, ethylhydroxy ethylcellulose, hydroxyethyl cellulose, carboxymethyl cellulose, sodium carboxymethyl cellulose, methyl cellulose, polyethylene oxides, polyvinyl alcohols, tragacanth, and xanthan. These polymers can be used alone or in mixtures with each other.

The amount of such polymers can vary between 15-80%. Other suitable excipients such as fillers, binders, and lubricants can be included in such sustain or delayed release formulations.

In another embodiment, suitable lubricant such as hydrogenated oil in combination with a binder forms a sustain release matrix capable of containing chloramphenicol compounds of the present invention and providing a sustained release of said compounds.

Compressed tablets can be sugar coated or film coated to mask any unpleasant taste and to protect the tablet from the atmosphere, or enteric coated for selective disintegration in the gastrointestinal tract. Technology for the formation of solid dosage forms such as capsules and compressed tablets, that utilize conventional pharmaceutical manufacturing equipment for their purpose, is described in detail in Remington's Pharmaceutical Sciences (Alfonso R. Gennaro ed., ch. 89, 18th ed. 1990).

There are three general methods of preparation of the materials to be included in the solid dosage form prior to compression: (1) dry granulation; (2) direct compression; and (3) wet granulation.

In a preferred methods of making the instant formulation, a dry granulation procedure is employed where all ingredients including chloramphenicol undergo a mixing, slugging, dry screening, lubricating and finally compressing phase. In the case of tablets, one of ordinary skill in the art can appreciate direct compression methodologies wherein the powdered material(s) to be included in the solid dosage form is compressed directly without modifying the physical nature of the material itself.

The wet granulation procedure includes mixing the powders to be incorporated into the dosage form in, e.g., a twin shell blender or double-cone blender and thereafter adding solutions of a binding agent to the mixed powders to obtain a granulation. Thereafter, the damp mass is screened, e.g., in a 6- or 8-mesh screen and then dried, e.g., via tray drying, the use of a fluid-bed dryer, spray-dryer, radio-frequency dryer, microwave, vacuum, or infra-red dryer.

The use of direct compression is limited to those situations where the drug or active ingredient has a requisite crystalline structure and physical characteristics required for formation of a pharmaceutically acceptable tablet. On the other hand, it is well known in the art to include one or more excipients which make the direct compression method applicable to drugs or active ingredients which do not possess the requisite physical properties. For solid dosage forms wherein the drug itself is to be administered in a relatively high dose (e.g., the drug itself comprises a substantial portion of the total tablet weight), it is necessary that the drug(s) itself have sufficient physical characteristics (e.g., cohesiveness) for the ingredients to be directly compressed. Typically, however, excipients are added to the formulation which impart good flow and compression characteristics to the material as a whole which is to be compressed. Such properties are typically imparted to these excipients via a pre-processing step such as wet granulation, slugging, spray drying, spheronization, or crystallization. Useful direct compression excipients include processed forms of cellulose, sugars, and dicalcium phosphate dihydrate, among others.

In general, wet granulation is a more preferred method over the dry granulation for preparing solid oral dosage forms. One of ordinary skill in the art would be able to recognize that the popularity of the wet granulation process as compared to the direct compression process is based on at least three advantages. First, wet granulation provides the material to be compressed with better wetting properties, particularly in the case of hydrophobic drug substances. The addition of a hydrophilic excipient makes the surface of a hydrophobic drug more hydrophilic, easing disintegration and dissolution. Second, the content uniformity of the solid dosage forms is generally improved.

Via the wet granulation method, all of the granules thereby obtained should contain approximately the same amount of drug. Thus, segregation of the different ingredients of the material to be compressed (due to different physical characteristics such as density) is avoided. Segregation is a potential problem with the direct compression method. Finally, the particle size and shape of the particles comprising the granulate to be compressed are optimized via the wet granulation process. This is due to the fact that when a dry solid is wet granulated, the binder “glues” particles together, so that they agglomerate in the granules which are more or less spherical.

In the instant case, one of ordinary skill in the art can appreciate that depending on the characteristics of the active ingredients, one could employ the most suitable process of preparing the final formulation. For example, in at least one embodiment of the instant invention, chloramphenicol may be combined with a second antibiotic such as TMP/SMX. In this aspect of the invention, suitable diluents, lubricants can be employed to form uniform granules comprising both chloramphenicol and TMP/SMX.

For at least one aspect of the instant invention, the wet granulation process may be employed in a manner that most of the components of the formulation, including the chloramphenicol drug and all or part of the diluent are formed into granules by the addition of a liquid, usually water, and optionally a binding agent. The remaining components such as the disintegrants and lubricants are then added and the blend tabletted. If color and/or flavors are used they may be added at any stage of the process. The second agents can also include such compounds that provides additional antimicrobial effects, or improves the absorption, distribution or side effect profile of chloramphenicol or analogues thereof.

The invention is illustrated by the following Examples.

### EXAMPLE 1

### Capsules Formulation Using Dry Granulation

Chloramphenicol is weight in amounts of about 30-75% w/w and then mixed with a suitable filler, a binder, a lubricant and a disintegrant. The resulting mixture is slugged, dried, milled, and screened before they are compacted into a capsule shelling.

### EXAMPLE 2

### Capsule Formulation Using Wet Granulation

Chloramphenicol and lactose and preferably a disintegrant are mixed initially and then wet granulated with a suitable aqueous solution. This wet mass is then dried in a fluidized bed, tray or other suitable dryer. The dried mixture may then be lubricated, filtered, milled, and/or granulated, to achieve the desirable and uniform particle size distribution. At the outset, the granules are blended with additional active ingredients or inactive excipients. This blend is then filled into capsule shelling.

### EXAMPLE 3

### Tablet Formulation Using Wet Granulation—

Chloramphenicol, lactose and an aliquot of vegetable oil are granulated with an aqueous solution of choice in a fluid bed granulator. The granules are dried and then blended with the remaining excipients and compressed into tablets.

### EXAMPLE 4

### Chloramphenicol Tablets—

In this embodiment tablets of chloramphenicol are manufactured by dry granulation using the following ingredients:

| | | |
|---|---|---|
| Chloramphenicol | 30-75% | w/w |
| Diluent | 10-30% | w/w |
| Non-polymeric Binder | 1-20% | w/w |
| Superdisintegrant | 1-15% | w/w |
| Flow agent | 0.1-5% | w/w |
| Polymeric binder | 0-10% | w/w |
| Lubricant | 0.5-10% | w/w |
| Optional second antibiotic | 5-40% | w/w |

### EXAMPLE 5

### Chloramphenicol Oral Pellet

In this embodiment pellets are placed into capsule shellings before oral administration. Each pellet comprise the following ingredients:

| Chloramphenicol | up to 70% | w/w |
|---|---|---|
| Diluent | 5-30% | w/w |
| Binder | 5-50% | w/w |
| Lubricant | 1-5% | w/w |
| Optional a second antibiotic | up to 40% | w/w |
| Optional a disintegrant | 0-10% | w/w |

### EXAMPLE 6

Chloramphenicol Capsule

In this embodiment capsule formulation comprise:

| | | |
|---|---|---|
| Chloramphenicol | 20 to 70% | w/w |
| Diluent | 10 to 30% | w/w |
| Lubricant | 1 to 5% | w/w |
| Optional second antibiotic | up to 40% | w/w |

### EXAMPLE 7

Chloramphenicol Capsule

In this embodiment capsules of chloramphenicol are manufactured by dry granulation. Each capsule contains:

| | |
|---|---|
| LevoChloramphenicol USP | 250 mg |
| Lactose NF Hydrous Capsuling Grade | 96.5 mg |
| Hydrogenated Vegetable Oil NF Lubritab | 11 mg |

What is claimed:

1. A method for reducing the resistance of an MRSA bacterium to an antibiotic selected from the group consisting of vancomycin and methicillin comprising administering to a patient in need thereof an effective amount of chloramphenicol or analogues thereof.

2. The method of claim 1, wherein said chloramphenicol is administered in doses of about 5 mg/kg/day to about 100 mg/kg/day to achieve a serum concentration of about 5-25 mg/liter.

3. The method of claim 2, wherein an additional antibiotic is added to the therapeutic regimen within 8 hours of the initiation of the therapy.

4. The method of claim 3, wherein the serum concentration of chloramphenicol is assessed every 3-5 half lives.

5. The method of 4, wherein side effect indices are monitored within 3-5 half lives of chloramphenicol doses.

6. A method for treating an MRSA resistant infection comprising administering to a patient in need thereof an effective amount of oral chloramphenicol or analogues thereof, wherein the oral dosage for is in the form of a tablet or capsule comprising about 70% w/w chloramphenicol.

7. The method of claim 6, further comprising administering a second antibiotic to the patient.

8. The method of claim 7, wherein the second antibiotic is selected from the group consisting of vancomycin, methicillin, penicillin, oxacillin, metronidazole, clindamycin, tetracycline, ciprofloxacin, gentamicin, tobramycin, doxycycline, trimethoprim/sulfamethoxazole, azithromycin, clarithromycin, roxithromycin, oleandomycin, spiramycin, josamycin, miocamycin, midecamycin, rosaramycin, troleandomycin, flurithromycin, rokitamycin or dirithromycin.

9. The method of claim 8, further comprising achieving a serum chloramphenicol concentration of about 5-25 mg/liter in said patient.

10. The method of claim 9, further comprising achieving a serum chloramphenicol concentration of about 5-12 mg/liter in said patient.

11. The method of claim 1, wherein said bacteria is found in blood, skin, urinary track, or abdomen.

12. The method of claim 10, wherein said bacteria is found in blood, skin, urinary track, or abdomen.

13. A method for reducing the resistance of a VRSA bacterium to an antibiotic selected from the group consisting of a vancomycin and methicillin comprising administering to a patient in need thereof an effective amount of chloramphenicol or analogues thereof.

14. The method of claim 13, wherein chloramphenicol is administered in doses of about 25 mg/kg/day to about 100 mg/kg/day to achieve a serum concentration of about 5-12 mg/liter.

15. The method of claim 13, wherein an additional antibiotic is added to the therapeutic regiment within 8 hours of the initiation of the therapy.

16. The method of claim 13, wherein the serum concentration of chloramphenicol is assessed every 3-5 half-lives.

17. The method of 13, wherein side effect indices are monitored within 5 half lives of chloramphenicol.

18. A method for treating an VRSA resistant infection comprising administering to a patient in need thereof an effective amount of an oral dosage form of chloramphenicol or analogues thereof, wherein the oral dosage form is in the form of a capsule comprising chloramphenicol 70% w/w.

19. The method of claim 18, further comprising hydrogenated cotton seed oil in amounts of about 3% w/w.

20. The method of claim 18, further comprising a second antibiotic to the patient.

21. The method of claim 18, further comprising achieving a serum concentration of about 25 mg/liter in said patient.

22. The method of claim 18, further comprising achieving a serum concentration of about 12 mg/liter in said patient.

23. An oral solid dosage formulation comprising chloramphenicol in amounts of about 10-75% w/w, a diluent in amounts of about 20-50% w/w, a lubricant in amounts of about 2.5-4% w/w and an optional second antibiotic up to 40% w/w.

24. The formulation of claim 23, wherein the chloramphenicol is levochloramphenicol.

25. The formulation of claim 23, wherein the diluent is selected from the group of microcrystalline cellulose, calcium hydrogen phosphate, lactose, hydrous lactose and mixtures thereof.

26. The formulation of claim 23, wherein the diluent is lactose NF hydrous capsuling grade, and the lubricant is hydrogenated vegetable oil NF lubritab.

27. An oral dosage formulation consisting essentially of levochloramphenicol USP 250 mg, lactose NF hydrous capsuling Grade 96.5 mg, hydrogenated vegetable oil NF lubritab 11 mg.

28. The formulation of claim 23, wherein of chloramphenicol size diameter of from about 100 μm to about 0.5 mm.

29. The formulation of claim 27, wherein of chloramphenicol size diameter of from about 100 μm to about 0.5 mm.

30. The formulation of claim 23, further comprising a disintegrant.
